(19) United States
(12) Patent Application Publication
(10) Pub. No.: US 2003/0175230 A1
(43) Pub. Date: Sep. 18, 2003

(54) Use and cosmetic compositions of starch betainates

(76) Inventor: Claude Dubief, Le Chesnay (FR)

Correspondence Address: D Douglas Price, Steptoe & Johnson, 1330 Connecticut Ave NW, Washington, DC, 20036, US

(21) Appl. No.: 10/333,207

(22) PCT Filed: Jul. 12, 2001
(86) PCT No.: PCT/FR01/02267

(30) Foreign Application Priority Data
Jul. 21, 2000 (FR) 00/09612

(51) Int. Cl. A61K 7/06; A61K 7/11; C08B 37/00

(52) U.S. Cl. 424/70.13; 536/18.7; 536/45

(57) ABSTRACT

The invention concerns a cosmetic use of at least a starch betainate for treating keratinous matter, in particular keratinous fibres and the skin, comprising said starch betainate combined with at least an agent beneficial for keratinous matter. The invention also concerns a cosmetic treatment method and a use of said composition, in particular as after-shampoo and hairstyling lotion.

The present invention relates to a cosmetic use of at least one starch betainate, to a composition for the cosmetic treatment of keratinous materials, in particular keratinous fibers and the skin, comprising this starch betainate combined with an agent beneficial for keratinous materials, to a method of cosmetic treatment and to a use of these compositions.

The hair is generally damaged and embrittled by the action of external atmospheric agents such as light and adverse weather conditions, and by mechanical or chemical treatments such as brushing, combing, bleaching, permanent waving and/or dyeing. The result is that the hair is often difficult to disentangle or to style, and the hair, even when abundant, retains with difficulty a style of good appearance because of the fact that the hair lacks liveliness and vitality.

Moreover, the skin may also be impaired to various degrees by the action of atmospheric agents and by the repeated action of detergent products. The skin tissues may be burnt, the skin becomes dry and rough and loses its natural elasticity. The appearance of dandruff, of an excess of seborrhea or conversely of excessive dryness may then be observed.

It is known to protect the hair and the skin from the effect of light by applying thereto polymeric or nonpolymeric, water-soluble or nonwater-soluble UV-screening agents, nanoparticles, antioxidants, metal complexing agents or chelators, or anti-free radical agents.

Embrittled hair may also be mechanically strengthened by applying thereto certain cationic polymers alone or as a mixture with specific electrolytes.

To better disentangle and prevent a certain roughness of the hair, it is also possible to apply thereto water-soluble or nonwater-soluble, monomeric or polymeric conditioners.

As regards the skin, dryness and roughness are combated by applying thereto moisturizing agents and/or emollients, and antidandruff and antisebborheic agents in order to combat skin disorders.

It is now known that these various raw materials conventionally used in cosmetics for treating hair and/or skin problems may be formulated as a mixture with cationic polymers in order to reinforce the effects and improve the cosmetic performance thereof. It is also possible to use for this purpose cationic starches which often appear in the list of prior art cationic polymers.

However, the prior art compositions make it possible to solve, to a certain degree, the problems raised, but their use is not satisfactory. Indeed, repeated applications of these compositions often have the effect of imparting an unpleasant feel to the hair, a loss of volume and of liveliness of the hair, and sometimes a lack of gloss.

Moreover, a persistent greasy feel is obtained on the skin during prolonged rinsing.

The applicant has surprisingly discovered that by using, in a cosmetically acceptable medium, at least one starch betainate in combination with at least one agent beneficial for keratinous materials, it was possible to avoid these undesirable effects.

The expression cosmetically acceptable medium is understood to mean a medium which is compatible with all keratinous materials such as the skin, the nails, the hair, the eyelashes and eyebrows, the lips and any other zone of the body and of the face, but which is also of pleasant odor, appearance and feel.

Agent beneficial for keratinous materials is an agent capable, in particular, of protecting, beautifying, conditioning, treating and/or maintaining in shape the keratinous materials, and in particular the hair.

Said starch betainates are novel polymers described in patent application WO 00/15669 from the company Raisio Chemicals OY and in the article “Preparation of starch betainate: a novel cationic starch derivative”. They are known only in the field of paper manufacture and have never been used in cosmetics.

These novel starch betainates are prepared from natural products and therefore have the advantage of being biodegradable. Combined with agents beneficial for keratinous materials, they make it possible to improve the performance of compositions for cosmetic treatment compared with combinations of cationic starches and of agents beneficial for keratinous materials of the prior art.

The subject of the present invention is therefore a cosmetic use of at least one starch betainate.

Another subject of the invention consists of a composition for the treatment of keratinous materials comprising, in a cosmetically acceptable medium, at least one starch betainate combined with an agent beneficial for keratinous materials.

The subject of the invention is also a method for the cosmetic treatment of the hair and the skin using the composition according to the invention.

Another subject of the invention consists in a use of the composition according to the invention as a shampoo or hair styling lotion.

Other subjects, characteristics, aspects and advantages of the invention will emerge even more clearly on reading the description and various examples which follow.

The present invention relates to the cosmetic use of at least one starch betainate corresponding to the following formula:

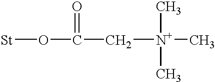

in which St represents a starch polymer structure.

The starch molecules may be derived from any plant source of starch such as, in particular corn, potato, oats, rice, tapioca, sorghum, barley or wheat. The starch is preferably derived from potato.

The starch betainates which can be used according to the present invention, and their preparation, are described in particular in application WO 00/15669 and in the article “Preparation of starch betainate: a novel cationic starch derivative”. They are prepared from natural products, namely a starch and a betaine as described above.

According to the invention, the composition for the cosmetic treatment of keratinous materials, in particular keratinous fibers and/or the skin, comprises, in a cosmetically acceptable medium, at least one starch betainate corresponding to the following formula:

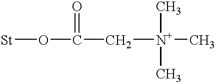

in which St represents a starch polymer structure, and at least one agent beneficial for keratinous materials.

The starch betainates are as described above and they are used in a quantity of between 0.01 and 20% by weight, preferably between 0.05 and 10% by weight, better still between 0.1 and 5% by weight relative to the total weight of the composition for cosmetic treatment.

Said agent beneficial for keratinous materials may be chosen from:

(1) saccharides, oligosaccharides, poly-saccharides which are hydrolyzed or nonhydrolyzed, modified or unmodified,

(2) amino acids, oligopeptides, peptides, proteins which are hydrolyzed or nonhydrolyzed, modified or unmodified,

(3) fatty acids and alcohols which are branched or unbranched,

(4) animal, vegetable or inorganic waxes,

(5) ceramides and pseudoceramides,

(6) hydroxylated organic acids,

(7) UV-screening agents,

(8) antioxidants and anti-free radical agents,

(9) chelators,

(10) antidandruff agents,

(11) seborrhea-regulating agents,

(12) soothing agents,

(13) cationic surfactants,

(14) cationic and amphoteric polymers,

(15) organomodified or nonorganomodified silicones,

(16) mineral, vegetable or animal oils,

(17) polyisobutenes and poly(α-olefins),

(18) esters,

(19) soluble or dispersed anionic polymers,

(20) soluble or dispersed nonionic polymers

and mixtures thereof.

The composition according to the invention may comprise, in addition, anionic, nonionic, amphoteric and/or zwitterionic surfactants well known in the art, or mixtures thereof, in a total quantity of less than 3% by weight relative to the total weight of the composition according to the invention.

The composition according to the invention may comprise one or more saccharides, oligosaccharides or polysaccharides which are hydrolyzed or nonhydrolyzed, modified or unmodified, identical or nonidentical. In general, compounds of this type, which can be used in the present invention, are chosen from those which are described in particular in “Encyclopedia of Chemical Technology, Kirk-Othmer, Third Edition, 1982, volume 3, pp. 896-900, and volume 15, pp. 439-458”, in “Polymers in Nature, by E. A. MacGREGOR and C. T. GREENWOOD, Editions John Wiley & Sons, Chapter 6, pp. 240-328, 1980” and in “Industrial Gums-Polysaccharides and their Derivatives, published by Roy L. WHISTLER, Second Edition, Edition Academic Press Inc.”, the content of these three books being fully included in the present application by way of reference.

By way of examples of saccharides, oligosaccharides and polysaccharides which are hydrolyzed or nonhydrolyzed, modified or unmodified, which can be used in the invention, there may be mentioned in particular glucans, modified or unmodified starches (such as those derived, for example, from cereals such as wheat, corn or rice, vegetables such as golden pea, tubers such as potato or cassava) which are different from starch betainate as described above, amylose, amylopectin, glycogen, dextrans, β-glucans, celluloses and derivatives thereof (methylcelluloses, hydroxy-alkylcelluloses, ethylhydroxyethylcelluloses, carboxy-methylcelluloses), fructosans, inulin, levan, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gums arabic, gums tragacanth, Ghatti gums, Karaya gums, carob gums, guar gums and xanthan gums, and mixtures thereof.

The composition according to the invention may comprise one or more amino acids, oligopeptides, peptides, proteins which are hydrolyzed or nonhydrolyzed, modified or unmodified. As amino acids, there may be mentioned, for example, cysteine, lysine, alanine, N-phenylalanine, arginine, glycine, leucine, and mixtures thereof. As oligopeptides, peptides, proteins which are hydrolyzed or nonhydrolyzed, modified or unmodified, which may be used in the composition according to the invention, there may be mentioned in particular hydrolyzates of wool or silk proteins, which are modified or unmodified, vegetable proteins such as wheat proteins.

The composition according to the invention may comprise one or more branched or unbranched fatty acids and alcohols. Among the fatty acids suitable for the present invention, there may be mentioned in particular C₈-C₃₀ carboxylic acids, such as palmitic acid, oleic acid, linoleic acid, myristic acid, stearic acid, lauric acid, and mixtures thereof. The fatty alcohols which can be used in the present invention comprise in particular C₈-C₃₀ alcohols such as, for example, palmityl, oleyl, linoleyl, myristyl, stearyl and lauryl alcohols, and mixtures thereof.

The composition according to the invention may comprise one or more animal, vegetable or mineral waxes.

A wax, for the purposes of the present invention, is a lipophilic compound, solid at room temperature (about 25° C.), with a reversible solid/liquid change of state, having a melting point greater than about 40° C. and which may be up to 200° C., and exhibiting, in the solid state, an anisotropic crystalline organization. In general, the size of the wax crystals is such that the crystals diffract and/or scatter light, conferring on the composition comprising them a cloudy appearance which is more or less opaque. By heating the wax to its melting point, it is possible to make it miscible with oils and to form a microscopically homogeneous mixture; but on bringing the temperature of the mixture back to room temperature, recrystallization of the wax from the oils of the mixture, which is microscopically and macroscopically detectable (opalescence), is obtained.

By way of waxes which can be used in the present invention, there may be mentioned waxes of animal origin such as beeswax, spermaceti, lanolin wax and lanolin derivatives; vegetable waxes such as Carnauba wax, Candelilla, Ouricury or Japan wax, cocoa butter or cork fiber or sugar cane waxes; mineral waxes, for example paraffin, petroleum jelly or lignite waxes or microcrystalline waxes or ozokerites, and mixtures thereof.

The composition according to the invention may comprise one or more ceramides and/or pseudoceramides. There may be mentioned in particular ceramides of classes I, II, III and V according to the DAWNING classification, and mixtures thereof, and more particularly N-oleyl-dehydrosphingosine.

The composition according to the invention may comprise one or more hydroxylated organic acids chosen from those which are well known and which are used in the art. There may be mentioned in particular citric acid, lactic acid, tartaric acid, malic acid and mixtures thereof.

The composition according to the invention may comprise one or more sunscreens active in UV-A and/or UV-B which are well known to persons skilled in the art. There may be mentioned in particular dibenzoylmethane derivatives such as 4-methyldibenzoylmethane, 4-isopropyl-dibenzoylmethane, 4-tert-butyldibenzoylmethane, 2,4-dimethyldibenzoylmethane, 4-tert-butyl-4′-diisopropyl-dibenzoylmethane, p-aminobenzoic acid and its esters such as 2-ethylhexyl p-dimethylaminobenzoate and N-propoxylated ethyl p-aminobenzoate, salicylates such as triethanolamine salicylate, cinnamic acid esters such as 2-ethylhexyl 4-methoxycinnamate, methyl diisopropyl-cinnamate, menthyl anthranilate, benzotriazole derivatives, triazine derivatives, β, β′-diphenylacrylate derivatives such as 2-ethylhexyl 2-cyano-3,3-diphenylacrylate and ethyl 2-cyano-3,3-diphenylacrylate, 2-phenylbenzimidazole-5-sulfonic acid and its salts, benzophenone derivatives, benzylidencamphor derivatives, silicone-based screening agents, and the like, and mixtures thereof.

As antioxidants and anti-free radical agents which can be used in the present invention, there may be mentioned, for example, ascorbic acid, ascorbylated compounds such as ascorbyl dipalmitate, t-butylhydroquinone, polyphenols such as fluoroglucinol, sodium sulfite, erythorbic acid, flavonoids, and mixtures thereof.

The composition according to the invention may comprise one or more chelators chosen in particular from EDTA (ethylenediaminetetraacetic acid) and its salts such as disodium EDTA and dipotassium EDTA, phosphated compounds such as sodium metaphosphate, sodium hexametaphosphate, tetrapotassium pyrophosphate, phosphonic acids and their salts such as the salts of ethylenediaminetetramethylenephosphonic acid, and mixtures thereof.

The composition according to the invention may comprise one or more antidandruff agents chosen, for example, from:

benzethonium chloride, benzalkonium chloride, chlorohexidine, chloramine T, chloramine B, 1,3-dibromo-5,5-dimethylhydantoin, 1,3-dichloro-5,5-dimethylhydantoin, 3-bromo-1-chloro-5,5-dimethyl-hydantoin, N-chlorosuccinimide;

1-hydroxy-2-pyridone derivatives such as, for example, 1-hydroxy-4-methyl-2-pyridone, 1-hydroxy-6-methyl-2-pyridone and 1-hydroxy-4,6-dimethyl-2-pyridone;

trihalocarbamides;

triclosan;

azole-containing compounds such as climbazole, ketaconazole, clotrinazole, econazole, isoconazole and miconazole b;

antifungal polymers such as amphotericin B or nystatin;

selenium sulfides;

sulfur in its various forms, cadmium sulfide, allantoin, coal or wood tars and derivatives thereof in particular, cade oil, undecylenic acid, fumaric acid, allylamines such as terbinafin;

or a mixture of these antidandruff agents.

They may also be used in the form of their addition salts with physiologically acceptable acids, in particular in the form of sulfuric, nitric, thiocyanic, hydrochloric, hydrobromic, hydroiodic, phosphoric, acetic, benzoic, glycolic, aceturic, succinic, nicotinic, tartaric, maleic, palmitic, methanesulfonic, propanoic, 2-oxopropanoic, propanedioic, 2-hydroxy-1,4-butanedionic, 3-phenyl-2-propenoic, α-hydroxybenzene-acetic, ethanesulfonic, 2-hydroxhanesulfonic, 4-methyl-benzenesulfonic, 4-amino-2-hydroxybenzoic, 2-phenoxy-benzoic, 2-acetyloxybenzoic, picric, lactic, citric, malic, and oxalic acids and amino acids.

The abovementioned antidandruff agents may also, where appropriate, be used in the from of their addition salts with physiologically acceptable organic or inorganic bases. Examples of organic bases are in particular low-molecular-weight alkanolamines such as ethanolamine, diethanolamine, N-ethylethanolamine, triethanolamine, diethylaminoethanol, 2-amino-2-methylpropanedione; nonvolatile bases such as ethylenediamine, hexamethylenediamine, cyclohexylamine, benzylamine, N-methylpiperazine; quaternary ammonium hydroxides, for example trimethylbenzyl hydroxide; guanidine and its derivatives, and particularly its alkylated derivatives. Examples of inorganic bases are in particular the salts of alkali metals, such as sodium or potassium; ammonium salts, the salts of alkaline-earth metals, such as magnesium or calcium; the salts of di-, tri- or tetravalent cationic metals, such as zinc, aluminum and zirconium. Alkanolamines, ethylenediamine and inorganic bases such as the alkali metal salts are preferred.

The composition according to the invention may comprise one or more seborrhea regulating agents such as succinylchitosan and poly-β-alanine, and mixtures thereof.

The composition according to the invention may comprise one or more soothing agents such as azulene and glycyrrhetinic acid, and mixtures thereof.

The composition according to the invention may comprise one or more cationic surfactants well known per se, such as the salts of optionally polyoxyalkylenated primary, secondary or tertiary fatty amines; quaternary ammonium salts such as tetraalkylammonium, alkyl-amidoalkyltrialkylammonium, trialkylbenzylammonium, trialkylhydroxyalkylammonium or alkylpyridinium chlorides or bromides; imidazoline derivatives; or amine oxides with a cationic character.

The composition according to the invention may comprise one or more cationic polymers. The expression “cationic polymer” is understood to mean any polymer containing cationic groups and/or groups which can be ionized to cationic groups.

The cationic polymers which can be used in accordance with the present invention may be chosen from all those already known per se to improve the cosmetic properties of hair treated with detergent compositions, namely in particular those described in patent application EP-A-0 337 354 and in French patent applications FR-A-2 270 846, 2 383 660, 2 598 611, 2 470 596 and 2 519 863.

The preferred cationic polymers are chosen from those which contain units comprising primary, secondary, tertiary and/or quaternary amine groups which may form part of the principal polymer chain, or which may be carried by a side substituent directly linked thereto.

The cationic polymers used generally have a number-average molecular mass of between about 500 and 5×10⁶, and preferably between about 10³ and 3×10⁶.

Among the cationic polymers, there may be mentioned more particularly polymers of the polyamine, polyaminoamide and poly(quaternary ammonium) type. They are known products.

The polymers of the polyamine, polyaminoamide and poly(quaternary ammonium) type which can be used in the composition of the present invention, are those described in French Patents Nos. 2 505 348 and 2 542 997. Among these polymers, there may be mentioned:

(1) the homopolymers or copolymers derived from esters or amides of acrylic or methacrylic acid;

(2) the cellulose ether derivatives comprising quaternary ammonium groups, described in French Patent 1 492 597;

(3) cationic cellulose derivatives such as cellulose copolymers or cellulose derivatives grafted with a quaternary ammonium water-soluble monomer, and described especially in U.S. Pat. No. 4,131,576, such as hydroxyalkyl celluloses like hydroxymethyl, hydroxyethyl or hydroxypropyl celluloses grafted especially with a methacryloylethyltrimethylammonium, methacrylamidopropyltrimethylammonium or dimethyl-diallylammonium salt;

(4) the cationic polysaccharides described more particularly in U.S. Pat. Nos. 3,589,578 and 4,031,307 such as guar gums containing cationic trialkylammonium groups;

(5) polymers consisting of piperazinyl units and of alkylene or hydroxyalkylene divalent groups with straight or branched chains, optionally interrupted by oxygen, sulfur or nitrogen atoms or by aromatic or heterocyclic rings, as well as the oxidation and/or quaternization products of these polymers. Such polymers are described especially in French Patent 2 162 025 and 2 280 361;

(6) water-soluble polyaminoamides, such as those described especially in French patents 2 252 840 and 2 368 508;

(7) polyaminoamide derivatives, for example adipic acid/dialkylaminohydroxyalkyldialkylenetriamine polymers in which the alkyl group comprises from 1 to 4 carbon atoms and preferably denotes a methyl, ethyl or propyl group and the alkylene group comprises from 1 to 4 carbon atoms, and preferably denotes the ethylene group. Such polymers are described especially in French Patent 1 583 363.

(8) polymers obtained by reaction of a polyalkylene-polyamine comprising two primary amine groups and at least one secondary amine group with a dicarboxylic acid chosen from diglycolic acid and saturated aliphatic dicarboxylic acids having from 3 to 8 carbon atoms. The molar ratio of the polyalkylene-polyamine to the dicarboxylic acid being between 0.8:1 and 1.4:1; the polyaminoamide resulting therefrom being made to react with epichlorohydrin in a molar ratio of epichlorohydrin relative to the secondary amine group of the polyaminoamide of between 0.5:1 and 1.8:1. Such polymers are described especially in American Patents 3 227 615 and 2 961 347;

(9) cyclopolymers of alkyldiallylamine or of dialkyldiallylammonium, such as the homopolymer of dimethyldiallylammonium chloride and the copolymers of diallyldimethylammonium chloride and of acrylamide;

(10)the quaternary diammonium polymers having a number-average molecular mass generally of between 1000 and 100,000, such as those described, for example in French Patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020;

(11) poly(quaternary ammonium) polymers such as those described especially in Patent application EP-A-122 324;

(12)quaternary vinylpyrrolidone and vinylimidazol polymers such as, for example, the products marketed under the names Luviquat® FC 905, FC 550 and FC 370 by the company B.A.S.F;

(13) polyamines such as Polyquart® H sold by HENKEL, referred to under the name of “POLYETHYLENEGLYCOL (15) TALLOW POLYAMINE” in the CTFA dictionary;

(14)the crosslinked polymers of methacryloyl-oxyalkyl (C₁-C₄ alkyl)tri(C₁-C₄ alkyl)ammonium salts such as those marketed under the name SALCARE® SC 92, SALCARE® SC 95 and SALCARE® SC 96 by the company ALLIED COLLOIDS; and mixtures thereof.

Other cationic polymers which can be used in the context of the invention are cationic proteins or hydrolyzates of cationic proteins, polyalkyleneimines, in particular polyethyleneimines, polymers containing vinylpyridine or vinylpyridinium units, condensates of polyamines and of epichlorohydrin, quaternary polyureylenes and chitin derivatives.

Among the abovementioned cationic polymers, which are suitable in the invention, it is preferable to use quaternary cellulose ether derivatives, cationic cyclopolymers, quaternary polymers of vinylpyrrolidone and of vinylimidazole, and mixtures thereof.

The composition according to the invention may comprise one or more amphoteric polymers.

The amphoteric polymers which can be used in accordance with the invention may be chosen from the polymers containing B and C units distributed statistically in the polymer chain where B denotes a unit which is derived from a monomer containing at least one basic nitrogen atom and C denotes a unit which is derived from an acidic monomer containing one or more carboxylic or sulfonic groups or alternatively B and C may denote groups which are derived from zwitterionic monomers of carboxybetaines or of sulfobetaines; B and C may also denote a cationic polymer chain containing primary, secondary, tertiary or quaternary amine groups, in which at least one of the amine groups carries a carboxylic or sulfonic group linked via a hydrocarbon group or alternatively B and C form part of a chain of a polymer with an α, β-dicarboxylic ethylene unit in which one of the carboxylic groups has been caused to react with a polyamine containing one or more primary or secondary amine groups.

The amphoteric polymers corresponding to the definition given above which are more particularly preferred are chosen from the following polymers:

(1) The polymers resulting from the copolymerization of a monomer derived from a vinyl compound carrying a carboxylic group such as more particularly acrylic acid, methacrylic acid, maleic acid, alpha-chloroacrylic acid, and of a basic monomer derived from a substituted vinyl compound containing at least one basic atom such as more particularly dialkylaminoalkyl methacrylate and acrylate, dialkylaminoalkyl-methacrylamide and acrylamide. Such compounds are described in American Patent No. 3 836 537. There may also be mentioned the sodium acrylate/acrylamido-propyltrimethylammonium chloride copolymer sold under the name POLYQUART⁷ KE 3033 by the company HENKEL.

The vinyl compound may also be a dialkyldiallylammonium salt such as diethyldiallylammonium chloride.

The copolymers of acrylic acid and of the latter monomer are provided under the names MERQUAT⁷ 280, MERQUAT⁷ 295 and MERQUAT⁷ PLUS 3330 by the company CALGON.

(2) The polymers containing units which are derived from:

a) at least one monomer chosen from acrylamides or methacrylamides substituted on the nitrogen by an alkyl group,

b) at least one acidic comonomer containing one or more reactive carboxylic groups, and

c) at least one basic comonomer such as esters with primary, secondary, tertiary and quaternary amine substituents of acrylic and methacrylic acids and the product of quaternization of dimethylaminoethyl meth-acrylate with dimethyl or diethyl sulfate.

The N-substituted acrylamides or methacrylamides more particularly preferred according to the invention are groups whose alkyl groups contain from 2 to 12 carbon atoms and more particularly N-ethylacrylamide, N-tert-butylacrylamide, N-tert-octylacrylamide, N-octylacryl-amide, N-decylacrylamide, N-dodecylacrylamide as well as the corresponding methacrylamides.

The acidic comonomers are chosen more particularly from acrylic, methacrylic, crotonic, itaconic, maleic and fumaric acids as well as the alkyl monoesters having 1 to 4 carbon atoms of maleic or fumaric anhydrides or acids. The basic comonomers preferred are methacrylates of aminoethyl, butylaminoethyl, N,N′-dimethyl-aminoethyl, N-tert-butylaminoethyl. Particularly used are the copolymers whose CTFA name (4th ed., 1991) is Octylacrylamide/acrylates/butylaminoethylmethacrylate copolymer such as the products sold under the name AMPHOMER⁷ or LOVOCRYL⁷ 47 by the company NATIONAL STARCH.

(3) The partially or completely alkylated and crosslinked polyaminoamides derived from polyaminoamides of general formula:

[CO—R₁₀—CO—Z] (II)

in which R₁₀ represents a divalent group derived from a saturated dicarboxylic acid, a mono- or dicarboxylic aliphatic acid with ethylenic double bond, an ester of a lower alkanol having 1 to 6 carbon atoms of these acids or a group which is derived from the addition of any one of said acids with a bis-primary or bis-secondary amine, and Z denotes a group which is derived from a bis-primary, mono- or bis-secondary polyalkylene-polyamine and preferably represents:

a) in the proportions of 60 to 100 mol%, the group

—NH[(CH₂)ₓ—NH]_{ₚ} (III)

where x=2 and p=2 or 3, or alternatively x=3 and p=2 this group being derived from diethylenetriamine, triethylenetetraamine or dipropylenetriamine;

b) in the proportions of 0 to 40 mol%, the group (III) above, in which x=2 and p=1 and which is derived from ethylenediamine, or the group which is derived from piperazine:

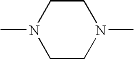

c) in the proportions of 0 to 20 mol%, the group —NH—(CH₂)₆—NH— which is derived from hexamethylenediamine, these polyamino amines being crosslinked by adding a bifunctional crosslinking agent chosen from the epihalohydrins, diepoxides, dianhydrides, bis-unsaturated derivatives, by means of 0.025 to 0.35 mol of crosslinking agent per amine group of the polyamino amide and alkylated by the action of acrylic acid, chloroacetic acid or of an alkanesultone or of their salts.

The saturated carboxylic acids are preferably chosen from the acids having 6 to 10 carbon atoms such as adipic, 2,2,4-trimethyladipic and 2,4,4-trimethyladipic acids, terephthalic acid, the acids with ethylene double bond such as for example acrylic, methacrylic and itaconic acids. The alkanesultones used in the alkylation are preferably propane- or butanesultone, the salts of the alkylating agents are preferably the sodium or potassium salts.

(4) The polymers containing zwitterionic units of formula:

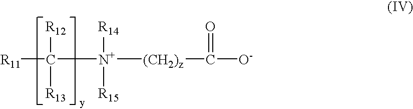

in which R₁₁ denotes a polymerizable unsaturated group such as an acrylate, methacrylate, acrylamide or methacrylamide group, y and z each represent an integer from 1 to 3, R₁₂ and R₁₃ represent a hydrogen atom, a methyl, ethyl or propyl group, R₁₄ and R₁₅ represent a hydrogen atom or an alkyl group such that the sum of the carbon atoms in R₁₄ and R₁₅ does not exceed 10.

The polymers comprising such units may also comprise units derived from nonzwitterionic monomers such as dimethyl- or diethylaminoethyl acrylate or methacrylate or alkyl acrylates or methacrylates, acrylamides or methacrylamides or vinyl acetate.

By way of example, there may be mentioned the copolymer of methyl methacrylate/methyl dimethylcarboxymethyl-ammonioethyl methacrylate such as the product sold under the name DIAFORMER⁷ Z301 by the company SANDOZ.

(5) The polymers derived from chitosan containing monomeric units corresponding to the following formulae:

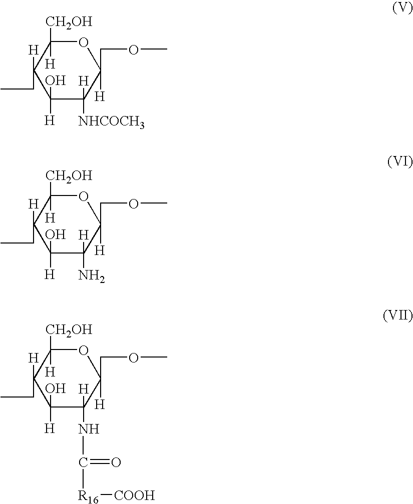

the (V) unit being present in proportions of between 0 and 30%, the (VI) unit in proportions of between 5 and 50% and the (VII) unit in proportions of between 30 and 90%, it being understood that in this unit F, R₁₆ represents a group of formula:

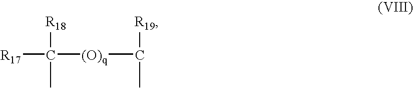

in which if q=0, R₁₇, R₁₈ and R₁₉, which are identical or different, each represent a hydrogen atom, a methyl, hydroxyl, acetoxy or amino residue, a monoalkylamine residue or a dialkylamine residue optionally interrupted by one or more nitrogen atoms and/or optionally substituted with one or more amine, hydroxyl, carboxyl, alkylthio or sulfonic groups, or an alkylthio residue whose alkyl group carries an amino residue, at least one of the R₁₇, R₁₈ and R₁₉ groups being in this case a hydrogen atom; or if q=1, R₁₇, R₁₈ and R₁₉ each represent a hydrogen atom, as well as the salts formed by these compounds with bases or acids.

(6) The polymers derived from the N-carboxyalkylation of chitosan such as N-carboxymethyl chitosan or N-carboxybutyl chitosan sold under the name EVALSAN⁷ by the company JAN DEKKER.

(7) The polymers corresponding to the general formula (IX) described for example in French Patent 1,400,366:

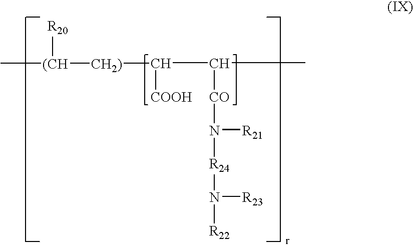

in which R₂₀ represents a hydrogen atom, a CH₃O, CH₃CH₂O or phenyl group, R21 denotes hydrogen or a lower alkyl group such as methyl or ethyl, R22 denotes hydrogen or a lower alkyl group such as methyl or ethyl, R₂₃ denotes a lower alkyl group such as methyl or ethyl or a group corresponding to the formula: —R₂₄—N(R₂₂)₂, R₂₄ representing a group —CH₂—CH₂—, —CH₂—CH₂—CH₂— or —CH₂—CH(CH₃)—, R₂₂ having the meanings mentioned above, as well as the higher homologues of these groups and containing up to 6 carbon atoms.

(8) Amphoteric polymers of the —D—X—D—X— type chosen from:

a) the polymers obtained by the action of chloroacetic acid or sodium chloroacetate on the compounds containing at least one unit of formula:

—D—X—D—X—D— (X)

where D denotes a group

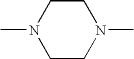

and X denotes the symbol E or E′, E or E′, which are identical or different, denote a bivalent group which is an alkylene group with a linear or branched chain containing up to 7 carbon atoms in the principal chain which is unsubstituted or substituted with hydroxyl groups and which may contain, in addition, oxygen, nitrogen or sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenyl-amine groups, or hydroxyl, benzylamine, amine oxide, quaternary ammonium, amide, imide, alcohol, ester and/or urethane groups.

b) The polymers of formula:

—D—X—D—X— (XI)

where D denotes a group

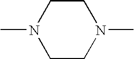

and X denotes the symbol E or E′ and, at least once, E′; E having the meaning indicated above and E′ is a bivalent group which is an alkylene group with a linear or branched chain having up to 7 carbon atoms in the principal chain, which is unsubstituted or substituted with one or more hydroxyl groups and containing one or more nitrogen atoms, the nitrogen atom being substituted with an alkyl chain optionally interrupted by an oxygen atom and necessarily containing one or more carboxyl functional groups or one or more hydroxyl functional groups and betainized by reaction with chloroacetic acid or sodium chloroacetate.

(9) The copolymers (C₁-C₅)alkyl vinyl ether/maleic anhydride partially modified by semiamidation with an N,N-dialkylaminoalkylamine such as N,N-dimethylamino-propylamine or by semiesterification with an N,N-dialkanolamine. These copolymers may also contain other vinyl comonomers such as vinylcaprolactam.

The amphoteric polymers which are particularly preferred according to the invention are those of the family (1).

The silicones which can be used in accordance with the invention may be soluble or insoluble in the composition and in particular may be polyorganosiloxanes insoluble in the composition of the invention; they may be provided in the form of oils, waxes, resins or gums.

The organopolysiloxanes are defined in greater detail in the book by Walter NOLL AChemistry and Technology of Silicones@ (1968) Academic Press. They may be volatile or nonvolatile.

When they are volatile, the silicones are more particularly chosen from those possessing a boiling point of between 60° C. and 260° C., and more particularly still from:

(i) cyclic silicones comprising from 3 to 7 silicon atoms, and preferably 4 to 5. They are, for example, the octamethylcyclotetrasiloxane marketed in particular under the name AVOLATILE SILICONE 7207″ by UNION CARBIDE or ASILBIONE 70045 V 2″ by RHODIA, the deca-methylcyclopentasiloxane marketed under the name AVOLATILE SILICONE 7158″ by UNION CARBIDE, ASILBIONE 70045 V 5″ by RHODIA, and mixtures thereof.

There may also be mentioned cyclocopolymers of the dimethylsiloxane/methylalkylsiloxane type, such as the ASILICONE VOLATILE FZ 3109″ marketed by the company UNION CARBIDE, having the chemical structure:

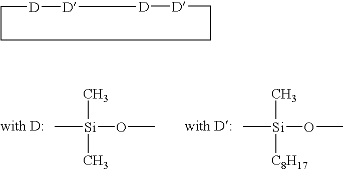

There may also be mentioned mixtures of cyclic silicones with organic compounds derived from silicon, such as the mixture of octamethylcyclotetrasiloxane and tetratrimethylsilylpentaerythritol (50/50) and the mixture of octamethylcyclotetrasiloxane and 1,1′-oxy(2,2,2′,2′,3,3′-hexatrimethylsilyloxy)bisneopentane;

(ii) linear volatile silicones having 2 to 9 silicon atoms and possessing a viscosity of less than or equal to 5×10⁻⁶ m²/s at 25° C. It is for example the decamethyltetrasiloxane marketed in particular under the name ASH 200″ by the company TORAY SILICONE. Silicones entering into this class are also described in the article published in Cosmetics and toiletries, Vol. 91, January 1976, p. 27-32 — TODD & BYERS AVolatile Silicone fluids for cosmetics@.

Among the nonvolatile silicones, there may be mentioned in particular polyalkylsiloxanes, polyarylsiloxanes, polyalkylarylsiloxanes, silicone gums and resins, polyorganosiloxanes modified by organofunctional groups, and mixtures thereof.

The organomodified silicones which can be used in accordance with the invention are silicones as defined above and comprising in their structure one or more organofunctional groups attached via a hydrocarbon group.

Among the organomodified silicones, there may be mentioned the polyorganosiloxanes comprising:

polyethyleneoxy and/or polypropyleneoxy groups optionally comprising C₆-C₂₄ alkyl groups such as the products called dimethicone-copolyol marketed by the company DOW CORNING under the name DC 1248 or the oils SILWET⁷ L 722, L 7500, L 77, L 711 from the company UNION CARBIDE and the (C₁₂)alkyl-methicone-copolyol marketed by the company DOW CORNING under the name Q2 5200;

substituted or unsubstituted amine-containing groups such as the products marketed under the name GP 4 Silicone Fluid and GP 7100 by the company GENESEE or the products marketed under the names Q2 8220 and DOW CORNING 929 or 939 by the company DOW CORNING. The substituted amine-containing groups are in particular C₁-C₄ aminoalkyl groups;

thiol groups, such as the products marketed under the names AGP 72 A@ and AGP 71@ from GENESEE;

alkoxylated groups, such as the product marketed under the name ASILICONE COPOLYMER F-755″ by SWS SILICONES and ABIL WAX⁷ 2428, 2434 and 2440 by the company GOLDSCHMIDT;

hydroxylated groups, such as the polyorgano-siloxanes with a hydroxyalkyl functional group which are described in French patent application FR-A-85 16334;

acyloxyalkyl groups such as, for example, the polyorganosiloxanes described in patent U.S. Pat. No. 4,957,732;

anionic groups of the carboxylic acid type, such as, for example, in the products described in patent EP 186 507 from the company CHISSO CORPORATION, or of the alkylcarboxylic type such as those present in the product X-22-3701E from the company SHIN-ETSU; 2-hydroxyalkylsulfonate; 2-hydroxyalkylthiosulfate such as the products marketed by the company GOLDSCHMIDT under the names AABIL⁷ S201@ and AABIL⁷ S255@;

hydroxyacylamino groups, such as the poly-organosiloxanes described in application EP 342 834. There may be mentioned, for example, the product Q2-8413 from the company DOW CORNING.

The composition according to the invention may comprise one or more mineral, vegetable or animal oils. There may be mentioned in particular as oils of vegetable origin, sweet almond oil, avocado oil, castor oil, olive oil, jojoba oil, sunflower oil, wheat germ oil, sesame oil, groundnut oil, grape seed oil, soya-bean oil, rapeseed oil, safflower oil, copra oil, maize oil, hazelnut oil, shea butter, palm oil, apricot stone oil, calophyllum oil; as oil of animal original perhydrosqualene; as oils of mineral origin, paraffin oil and liquid paraffin; and mixtures thereof.

The composition according to the invention may comprise one or more polyisobutenes and poly(α-olefins), chosen from those which are well known in the art.

The composition according to the invention may comprise one or more esters. As examples, there may be mentioned in particular fatty acid esters such as isopropyl myristate, isopropyl palmitate, 2-ethylhexyl palmitate, Purcellin oil (stearyl octanoate), isononyl or isostearyl isononanoate, isopropyl lanolate, and mixtures thereof.

The composition according to the invention may comprise one or more soluble or dispersed anionic polymers, well known per se. The anionic polymers generally used in the present invention are polymers comprising groups derived from carboxylic, sulfonic or phosphoric acids, and having a weight-average molecular mass of between 500 and 5 000 000.

The carboxylic groups are provided by unsaturated mono-or dicarboxylic acid monomers such as those corresponding to the formula:

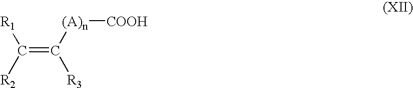

in which n is an integer from 0 to 10, A denotes a methylene group, optionally linked to the carbon atom of the unsaturated group or to the neighboring methylene group when n is greater than 1, via a heteroatom such as oxygen or sulfur, R₁ denotes a hydrogen atom, or a phenyl or benzyl group, R₂ denotes a hydrogen atom, a carboxyl or lower alkyl group, R₃ denotes a hydrogen atom or a lower alkyl group, a group —CH₂—COOH, or a phenyl or benzyl group.

In the above formula (XII), a lower alkyl group preferably comprises from 1 to 4 carbon atoms and denotes in particular the methyl and ethyl groups.

The anionic polymers with carboxylic groups preferred according to the invention are:

A) The homo- or copolymers of acrylic or methacrylic acid or their salts and in particular the products sold under the names VERSICOL⁷ E or K by the company ALLIED COLLOID and ULTRAHOLD⁷ by the company BASF, the copolymers of acrylic acid and of acrylamide sold in the form of their sodium salt under the names RETEN⁷ 421, 423 or 425 by the company HERCULES, the sodium salts of the polyhydroxycarboxylic acids;

B) The copolymers of acrylic or methacrylic acids with a monoethylene monomer such as ethylene, styrene, vinyl esters, acrylic or methacrylic acid esters, optionally grafted onto a polyalkylene glycol such as polyethylene glycol and optionally crosslinked. Such polymers are described in particular in French Patent 1,222,944 and German Application 2,330,956, copolymers of this type containing in their chain an acrylamide unit optionally N-alkylated and/or hydroxyalkylated as described especially in Luxembourg Patent applications 75370 and 75371 or offered under the name QUADRAMER⁷ by the company AMERICAN CYANAMID. There may also be mentioned the copolymers of acrylic acid and C₁-C₄ alkyl methacrylate and the copolymer of methacrylic acid and ethyl acrylate sold under the name LUVIMER⁷ MAEX by the company BASF.

C) The copolymers derived from crotonic acid such as those containing in their chain vinyl propionate or acetate units and optionally other monomers such as methallyl or allyl esters, vinyl ether or vinyl ester of a linear or branched saturated carboxylic acid with a long hydrocarbon chain such as those containing at least 5 carbon atoms, it being possible for these polymers to be optionally grafted and crosslinked or alternatively a vinyl, allyl or methallyl ester of an α- or β-cyclic carboxylic acid. Such polymers are described, inter alia, in French Patents 1,222,944; 1,580,545; 2,265,782; 2,265,781; 1,564,110 and 2,439,798. Commercial products entering into this class are the resins 28-29-30, 26-13-14 and 28-13-10 sold by the company NATIONAL STARCH.

D) The polymers derived from maleic, fumaric or itaconic acids or anhydrides and vinyl esters, vinyl ethers, vinyl halides, phenylvinyl derivatives, acrylic acid and its esters; these polymers may be esterified. Such polymers are described in particular in patents U.S. Pat. Nos. 2,047,398, 2,723,248, 2,102,113, patent GB 839 805, and in particular those sold under the names GANTREZ⁷ AN or ES by the company ISP.

The polymers also entering into this class are the copolymers of maleic, citraconic or itaconic anhydrides and an allyl or methallyl ester optionally comprising an acrylamide or methacrylamide group, an α-olefin, acrylic or methacrylic esters, acrylic or methacrylic acids or vinylpyrrolidone in their chain; the anhydride functional groups are monoesterified or monoamidated. These polymers are for example described in French patents 2 350 384 and 2 357 241 by the applicant.

E) The polyacrylamides containing carboxylate groups.

The polymers comprising sulfonic groups are polymers containing vinylsulfonic, styrenesulfonic, naphthalene-sulfonic or acrylamidoalkylsulfonic units.

These polymers may be especially chosen from:

the salts of polyvinylsulfonic acid having a molecular mass of between about 1000 and 100,000 as well as the copolymers with an unsaturated comonomer such as acrylic or methacrylic acids and their esters as well as acrylamide or its derivatives, vinyl ethers and vinylpyrrolidone;

the salts of polystyrenesulfonic acid, the sodium salts having a molecular mass of about 500,000 and about 100,000 sold respectively under the names Flexan⁷ 500 and Flexan⁷ 130 by National Starch. These compounds are described in Patent FR 2,198,719;

the salts of polyacrylamidesulfonic acids, such as those mentioned in Patent U.S. Pat. No. 4,128,631 and more particularly polyacrylamidoethylpropanesulfonic acid sold under the name COSMEDIA POLYMER⁷ HSP 1180 by Henkel.

According to the invention, the anionic polymers are preferably chosen from the acrylic acid copolymers such as the terpolymer acrylic acid/ethyl acrylate/N-tert-butylacrylamide sold under the name ULTRAHOLD⁷ STRONG by the company BASF, the copolymers derived from crotonic acid such as the terpolymers vinyl acetate/vinyl tert-butylbenzoate/crotonic acid and the terpolymers crotonic acid/vinyl acetate/vinyl neododecanoate sold under the name Résine 28-29-30 by the company NATIONAL STARCH, the polymers derived from itaconic, fumaric and maleic acids or anhydrides with vinyl esters, vinyl ethers, vinyl halides, phenylvinyl derivatives, acrylic acid and its esters such as the monoesterified maleic anhydride/methylvinyl ether copolymer sold under the name GANTREZ⁷ ES 425 by the company ISP, the copolymers of methacrylic acid and of methyl methacrylate sold under the name EUDRAGIT⁷ L by the company ROHM PHARMA, the copolymer of methacrylic acid and of ethyl acrylate sold under the name LUVIMER⁷ MAEX by the company BASF, the copolymer vinyl acetate/crotonic acid sold under the name LUVISET⁷ CA 66 by the company BASF and the terpolymer vinyl acetate/crotonic acid/polyethylene glycol under the name ARISTOFLEX⁷ A by the company BASF.

The anionic polymers most particularly preferred are those chosen from the monoesterified maleic anhydride/methylvinyl ether copolymer sold under the name GANTREZ⁷ ES 425 by the company ISP, the copolymers of methacrylic acid and of methyl methacrylate sold under the name EUDRAGIT⁷ L by the company ROHM PHARMA, the copolymer of methacrylic acid and ethyl acrylate sold under the name LUVIMER⁷ MAEX by the company BASF, the terpolymer vinylpyrrolidone/acrylic acid/lauryl methacrylate sold under the name ACRYLIDONE⁷ LM by the company ISP.

According to the invention, the anionic polymers may also be used in the form of a latex or pseudolatex, that is to say in the form of an aqueous dispersion of insoluble polymer particles.

According to the invention, the anionic polymer(s) may represent from 0.1% to 20% by weight, preferably from 0.2% to 15% by weight, and still more preferably from 0.5% to 10% by weight, of the total weight of the final composition.

The composition according to the invention may comprise one or more soluble or dispersed nonionic polymers. As nonionic polymers which can be used according to the present invention, there may be mentioned in particular:

homopolymers of vinylpyrrolidone;

copolymers of vinylpyrrolidone and vinyl acetate;

polyalkyloxazolines such as the polyethyloxazolines provided by the company DOW CHEMICAL under the names PEOX® 50 000, PEOX® 200 000 and PEOX® 500 000;

homopolymers of vinyl acetate such as the product provided under the name APPRETAN® EM by the company HOECHST or the product provided under the name RHODOPAS® A 012 by the company RHONE POULENC;

copolymers of vinyl acetate and an acrylic ester such as the product provided under the name RHODOPAS® AD 310 from RHONE POULENC;

copolymers of vinyl acetate and ethylene such as the product provided under the name APPRETAN® TV by the company HOECHST;

copolymers of vinyl acetate and a maleic ester, for example of dibutyl maleate such as the product provided under the name APPRETAN® MB EXTRA by the company HOECHST;

copolymers of polyethylene and maleic anhydride;

homopolymers of alkyl acrylates and homopolymers of alkyl methacrylates such as the product provided under the name MICROPEARL® RQ 750 by the company MATSUMOTO or the product provided under the name LUHYDRAN® A 848 S by the company BASF;

copolymers of acrylic esters such as, for example, copolymers of alkyl acrylates and alkyl methacrylates such as the products provided by the company ROHM & HAAS under the names PRIMAL® AC-261 K and EUDRAGIT® NE 30 D, by the company BASF under the names ACRONAL® 601, LUHYDRAN® LR 8833 or 8845, or by the company HOECHST under the names APPRETAN® N 9213 or N921 2;

copolymers of acrylonitrile and a nonionic monomer chosen, for example, from butadiene and alkyl (meth)acrylates; there may be mentioned the products provided under the name NIPOL® LX 531 8 by the company NIPPON ZEON or those provided under the name CJ 0601 8 by the company ROHM & HAAS;

polyurethanes such as the products provided under the names ACRYSOL® RM 1020 or ACRYSOL® RM 2020 by the company ROHM & HAAS, the products URAFLEX® XP 401 UZ, URAFLEX® XP 402 UZ by the company DSM RESINS;

copolymers of alkyl acetate and urethane such as the product 8538-33 by the company NATIONAL STARCH;

polyamides such as the product ESTAPOR® LO 11 provided by the company RHONE POULENC;

chemically modified or unmodified nonionic guar gums.

The unmodified nonionic guar gums are, for example, the products sold under the name VIDOGUM® GH 175 by the company UNIPECTINE or under the name JAGUAR® C. by the company MEYHALL.

The modified nonionic guar gums which can be used according to the invention are preferably modified by C₁-C₆ hydroxyalkyl groups. There may be mentioned, by way of example, hydroxymethyl, hydroxyethyl, hydroxypropyl and hydroxybutyl groups.

These guar gums are well known in the state of the art and may, for example, be prepared by reacting corresponding alkene oxides, such as, for example, propylene oxides, with guar gum so as to obtain a guar gum modified by hydroxypropyl groups.

Such nonionic guar gums optionally modified by hydroxyalkyl groups are, for example, sold under the tradenames JAGUAR® HP8, JAGUAR® HP60 and JAGUAR® HP120, JAGUAR® DC 293 and JAGUAR® HP 105 by the company MEYHALL, or under the name GALACTASOL® 4H4FD2 by the company AQUALON.

The alkyl groups of the nonionic polymers preferably comprise from 1 to 6 carbon atoms.

The composition for treating keratinous materials according to the invention may contain one or more agents beneficial for keratinous materials, described above, in a total quantity of between 0.01 and 30% by weight, preferably between 0.05 and 25% by weight, better still between 0.1 and 20% by weight, relative to the total weight of the composition for cosmetic treatment.

The cosmetically acceptable medium may consist solely of water or of a mixture of water and a cosmetically acceptable solvent such as C₁-C₄ lower alcohol, such as ethanol, isopropanol, tert-butanol, n-butanol; alkylene glycols such as propylene glycol, and glycol ethers.

The pH of the compositions of the invention is between 4 and 8, preferably between 5 and 7.

The compositions according to the invention may also contain additives such as associative or non-associative, anionic, amphoteric, zwitterionic, nonionic or cationic, natural or synthetic polymeric thickeners, nonpolymeric thickeners such as acids or electrolytes, pearlescent agents, opacifiers, organic solvents, perfumes, colorants, inorganic or organic particles, preservatives, pH-stabilizing agents.

Persons skilled in the art will be careful to choose the optional additives and their quantity so that they do not damage the properties of the treatment compositions.

These additives are present in the composition according to the invention in a quantity ranging from 0 to 20% by weight relative to the total weight of the composition.

The compositions may be provided in the form of fluid or thickened liquids, gels, creams, foams, waxes, W/O, O/W emulsions or multiple emulsions.

They may be used as shampoos, rinse-out or leave-in treatments, deep treatment masks, lotions or creams for treating the scalp, mascaras, makeup removers, products for dyeing the hair or coloring the skin, sun protection products, and the like.

The present invention also relates to a method of cosmetic treatment which consists in applying an effective quantity of a composition as described above, to the hair, in carrying out an optional rinsing after an optional exposure time.

According to a preferred embodiment of the invention, the composition may be used as after-shampoo or as hair styling lotion.

The following examples illustrate the present invention and should not be considered in any manner as limiting the invention.

### EXAMPLE 1

An after-shampoo according to the invention is prepared from the following ingredients:

| | | |
|---|---|---|
| Starch betainate having a degree of | 0.5 | g |
| substitution of 0.8 | | |
| Behenyltrimethylammonium chloride | 1.5 | g |
| Mixture (80/20) of cetylstearyl alcohol and | 4 | g |
| oxyethylenated cetylstearyl alcohol (33 | | |
| oxyethylene units) | | |
| Water qs | 100 | g |

After rinsing, the hair is soft and easy to disentangle.

### EXAMPLE 2

An after-shampoo according to the invention is prepared from the following ingredients:

| | | |
|---|---|---|
| Starch betainate having a degree of | 0.5 | g |
| substitution of 0.8 | | |
| Behenyltrimethylammonium chloride | 1.5 | g |
| N-oleyldihydrosphingosine | 0.5 | |
| Water qs | 100 | g |

After rinsing, the hair is smooth and easy to disentangle.

### EXAMPLE 3

A hairstyling lotion is prepared from the following ingredients:

| | | |
|---|---|---|
| Starch betainate having a degree of | 0.5 | g |
| substitution of 0.8 | | |
| Vinylcaprolactam/vinylpyrrolidone/dimethyl- | 0.5 | g |
| aminoethyl methacrylate terpolymer, as a 37% | | |
| solution in ethanol and sold under the name | | |
| ADVANTAGE ® HC 37 by ISP | | |
| Water, perfume, preservative qs | 100 | g |
| pH 6 | | |

After drying, the hair is soft, lively and easy to style.

1. The cosmetic use of at least one starch betainate corresponding to the following formula: 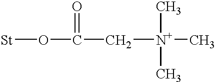
in which St represents a starch polymer structure.

2. The use as claimed in claim 1, characterized in that the starch is derived from corn, potato, oats, rice, tapioca, sorghum, barley or wheat.

3. A composition for the cosmetic treatment of keratinous materials, characterized in that it comprises at least one starch betainate corresponding to the following formula: 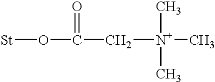
in which St represents a starch polymer structure, and at least one agent beneficial for keratinous materials.

4. The composition as claimed in claim 3, characterized in that the starch is derived from corn, potato, oats, rice, tapioca, sorghum, barley or wheat.

5. The composition as claimed in claim 3 or 4, characterized in that the agent beneficial for keratinous materials is chosen from:
(1) saccharides, oligosaccharides, polysaccharides which are hydrolyzed or nonhydrolyzed, modified or unmodified,
(2) amino acids, oligopeptides, peptides, proteins which are hydrolyzed or nonhydrolyzed, modified or unmodified,
(3) fatty acids and alcohols which are branched or unbranched,
(4) animal, vegetable or inorganic waxes,
(5) ceramides and pseudoceramides,
(6) hydroxylated organic acids,
(7) UV-screening agents,
(8) antioxidants and anti-free radical agents,
(9) chelators,
(10) antidandruff agents,
(11) seborrhea-regulating agents,
(12) soothing agents,
(13) cationic surfactants,
(14) cationic and amphoteric polymers,
(15) organomodified or nonorganomodified silicones,
(16) mineral, vegetable or animal oils,
(17) polyisobutenes and poly(α-olefins),
(18) esters,
(19) soluble or dispersed anionic polymers,
(20) soluble or dispersed nonionic polymers and mixtures thereof.

6. The composition as claimed in any one of claims 3 to 5, characterized in that it comprises, in addition, anionic, nonionic, amphoteric and/or zwitterionic surfactants, or mixtures thereof, in a total quantity of less than 3% by weight relative to the total weight of the composition.

7. The composition as claimed in claim 5, characterized in that the saccharides, oligosaccharides and polysaccharides which are hydrolyzed or nonhydrolyzed, modified or unmodified, are chosen from glucans, modified or unmodified starches which are different from said starch betainate, amylose, amylopectin, glycogen, dextrans, β-glucans, celluloses and derivatives thereof, fructosans, inulin, levan, mannans, xylans, lignins, arabans, galactans, galacturonans, chitin, glucoronoxylans, arabinoxylans, xyloglucans, galactomannans, glucomannans, pectic acids and pectins, alginic acid and alginates, arabinogalactans, carrageenans, agars, glycosaminoglucans, gums arabic, gums tragacanth, Ghatti gums, Karaya gums, carob gums, guar gums and xanthan gums, and mixtures thereof.

8. The composition as claimed in claim 5, characterized in that the amino acids, oligopeptides, peptides, proteins which are hydrolyzed or nonhydrolyzed, modified or unmodified, are chosen from cysteine, lysine, alanine, N-phenylalanine, arginine, glycine, leucine, and mixtures thereof.

9. The composition as claimed in claim 5, characterized in that the branched or unbranched fatty acids and alcohols comprise from 8 to 30 carbon atoms and are chosen from palmitic acid, oleic acid, linoleic acid, myristic acid, stearic acid, lauric acid, and palmityl, oleyl, linoleyl, myristyl, stearyl and lauryl alcohols, and mixtures thereof.

10. The composition as claimed in claim 5, characterized in that the animal, vegetable or mineral waxes are chosen from beeswax, spermaceti, lanolin wax and lanolin derivatives; Carnauba, Candelilla, Ouricury or Japan wax, cocoa butter or cork fiber or sugar cane waxes; paraffin, petroleum jelly or lignite waxes or microcrystalline waxes or ozokerites, and mixtures thereof.

11. The composition as claimed in claim 5, characterized in that the ceramides and pseudoceramides are chosen from the ceramides of classes I, II, III and V according to the Dawning classification, and mixtures thereof.

12. The composition as claimed in claim 5, characterized in that the hydroxylated organic acids are chosen from citric acid, lactic acid, tartaric acid, malic acid and mixtures thereof.

13. The composition as claimed in claim 5, characterized in that the UV-screening agents are chosen from dibenzoylmethane derivatives, p-amino-benzoic acid and its esters, salicylates, cinnamic acid esters, benzotriazole derivatives, triazine derivatives, β, β′-diphenylacrylate derivatives, 2-phenylbenzimidazole-5-sulfonic acid and its salts, benzophenone derivatives, benzylidencamphor derivatives, silicone-based screening agents, and mixtures thereof.

14. The composition as claimed in claim 5, characterized in that the antioxidants and anti-free radical agents are chosen from ascorbic acid, ascorbylated compounds, t-butylhydroquinone, polyphenols, sodium sulfite, erythorbic acid, flavonoids, and mixtures thereof.

15. The composition as claimed in claim 5, characterized in that the chelators are chosen from EDTA and its salts, phosphated compounds, phosphonic acids and their salts, and mixtures thereof.

16. The composition as claimed in claim 5, characterized in that the antidandruff agents are chosen from:
benzethonium chloride, benzalkonium chloride, chlorohexidine, chloramine T, chloramine B, 1,3-dibromo-5,5-dimethylhydantoin, 1,3-dichloro-5,5-dimethylhydantoin, 3-bromo-1-chloro-5,5-dimethyl-hydantoin, N-chlorosuccinimide;
1-hydroxy-2-pyridone derivatives;
trihalocarbamides;
triclosan;
azole-containing compounds;
antifungal polymers;
selenium sulfides;
sulfur in various forms;
or a mixture of these antidandruff agents.

17. The composition as claimed in claim 5, characterized in that the seborrhea regulating agents are chosen from succinylchitosan and poly-β-alanine, and mixtures thereof.

18. The composition as claimed in claim 5, characterized in that the soothing agents are chosen from azulene and glycyrrhetinic acid, and mixtures thereof.

19. The composition as claimed in claim 5, characterized in that the cationic surfactants are chosen from the salts of optionally polyoxyalkylenated primary, secondary or tertiary fatty amines; quaternary ammonium salts such as tetraalkylammonium, alkyl-amidoalkyltrialkylammonium, trialkylbenzylammonium, trialkylhydroxyalkylammonium or alkylpyridinium chlorides or bromides; imidazoline derivatives; or amine oxides with a cationic character; and mixtures thereof.

20. The composition as claimed in claim 5, characterized in that the cationic polymers are chosen from quaternary cellulose ether derivatives, cationic cyclopolymers, cationic polysaccharides, quaternary polymers of vinylpyrrolidone and vinylimidazole, and mixtures thereof.

21. The composition as claimed in claim 5, characterized in that the organomodified or nonorganomodified silicones are soluble or insoluble in the composition and are chosen from volatile silicones such as cyclic silicones comprising from 3 to 7 silicon atoms, linear silicones having 2 to 9 silicon atoms and possessing a viscosity of less than or equal to 5×10⁻⁶ m²/s at 25° C.; nonvolatile silicones such as polyalkylsiloxanes, polyarylsiloxanes, polyalkylaryl-siloxanes, silicone gums and resins, polyorgano-siloxanes modified by organofunctional groups such as polyethyleneoxy and/or propyleneoxy groups, substituted or unsubstituted amine-containing groups, thiol, alkoxylated or hydroxylated groups, acyloxyalkyl, carboxylic acid or hydroxyacylamino groups; and mixtures thereof.

22. The composition as claimed in claim 5, characterized in that the mineral, vegetable or animal oils are chosen from paraffin oil, liquid paraffin, sweet almond oil, avocado oil, castor oil, olive oil, jojoba oil, sunflower oil, wheat germ oil, sesame oil, groundnut oil, grape seed oil, soya-bean oil, rapeseed oil, safflower oil, copra oil, maize oil, hazelnut oil, shea butter, palm oil, apricot stone oil, calophyllum oil, perhydrosqualene, and mixtures thereof.

23. The composition as claimed in claim 5, characterized in that the esters are chosen from fatty acid esters such as isopropyl myristate, isopropyl palmitate, 2-ethylhexyl palmitate, Purcellin oil (stearyl octanoate), isononyl or isostearyl isononanoate, isopropyl lanolate, and mixtures thereof.

24. The composition as claimed in claim 5, characterized in that the soluble or dispersed anionic polymers are chosen from polymers comprising groups derived from carboxylic, sulfonic or phosphoric acids, and having a weight-average molecular mass of between 500 and 5 000 000; and mixtures thereof.

25. The composition as claimed in claim 5, characterized in that the soluble or dispersed nonionic polymers are chosen from homopolymers of vinyl-pyrrolidone; copolymers of vinylpyrrolidone and vinyl acetate; polyalkyloxazolines; homopolymers of vinyl acetate; copolymers of vinyl acetate and an acrylic ester; copolymers of vinyl acetate and ethylene; copolymers of vinyl acetate and a maleic ester; copolymers of polyethylene and maleic anhydride; homopolymers of alkyl acrylates and homopolymers of alkyl methacrylates; copolymers of acrylic esters; copolymers of acrylonitrile and butadiene or alkyl (meth)acrylate; polyurethanes; copolymers of alkyl acetate and urethane; polyamides; nonionic guar gums which are chemically modified or unmodified; and mixtures thereof.

26. The composition as claimed in any one of claims 3 to 25, characterized in that it comprises starch betainate in a quantity between 0.01 and 20% by weight, and the agent beneficial for keratinous materials in a quantity between 0.01 and 30% by weight, relative to the total weight of the composition.

27. The composition as claimed in any one of claims 3 to 26, characterized in that it comprises, in addition, additives such as thickeners, pearlescent agents, opacifiers, organic solvents, perfumes, colorants, inorganic or organic particles, preservatives and pH-stabilizing agents.

28. The composition as claimed in any one of claims 3 to 27, characterized in that it is provided in the form of a shampoo.

29. A method of cosmetic treatment, characterized in that a composition as claimed in any one of claims 3 to 28 is applied to the hair.

30. The use of a composition as claimed in any one of claims 3 to 28, as after-shampoo.

31. The use of a composition as claimed in any one of claims 3 to 28, as hairstyling lotion.
